SEQUENCE LISTING (1 sequence)

SEQ ID NO: 1 - protein, 5 aa, ARTIFICIAL SEQUENCE
  Recombinanat human furin protein substrate
ERTKR

(19) United States
(12) Patent Application Publication
(10) Pub. No.: US 2024/0108678 A1
(43) Pub. Date: Apr. 4, 2024

(54) ANTI-VIRAL COMPOSITIONS

(71) Applicants: Muhammed Majeed, Bangalore (IN); Kalyanam Nagabhushanam, East Windsor, NJ (US); Lakshmi Mundkur, Bangalore (IN)

(72) Inventors: Muhammed Majeed, Bangalore (IN); Kalyanam Nagabhushanam, East Windsor, NJ (US); Lakshmi Mundkur, Bangalore (IN)

(21) Appl. No.: 18/265,899

(22) PCT Filed: Dec. 16, 2021
(86) PCT No.: PCT/US21/63420

§ 371 Date: Jun. 7, 2023

Related U.S. Application Data
(60) Provisional application No. 63/125,572, filed on Dec. 15, 2020.

(51) Int. Cl. A61K 36/67; A61K 31/05; A61K 31/122; A61K 31/198; A61K 31/30; A61K 31/315; A61K 31/375; A61K 31/4415; A61K 31/4525; A61K 31/525; A61K 31/593; A61K 36/19; A61K 36/704; A61K 36/71; A61P 31/14

(52) U.S. Cl. CPC A61K 36/67; A61K 31/05; A61K 31/122; A61K 31/198; A61K 31/30; A61K 31/315; A61K 31/375; A61K 31/4415; A61K 31/4525; A61K 31/525; A61K 31/593; A61K 36/19; A61K 36/704; A61K 36/71; A61P 31/14

(57) ABSTRACT

The present invention discloses methods and compositions comprising. Andrographis paniculata extract standardized to contain not less than 10% w/w andrographolides, Polygonum cuspidatum extract standardized to contain not less than 80% w/w resveratrol, Piper nigrum extract standardized to contain 95% w/w piperine, 95% tetrahydrocurcuminoids, vitamins for the management of viral infections, specifically COVID infection. The invention also discloses the use of the compositions in preventing proteolytic activation of viral spike protein by inhibiting the furin enzyme.

### CROSS REFERENCE TO RELATED APPLICATIONS

This is a US national phase application of PCT application no PCT/US21/63420, filed on 16 Dec. 2021 claiming priority form U.S. provisional application No. 63/125,572 filed on 15 Dec. 2020, the contents of which are incorporated herein by reference.

### FIELD OF INVENTION

The invention in general relates to compositions for preventing viral infections. Specifically, the invention discloses the anti-viral and immune modulatory potential of one or more compositions comprising ore or more ingredients selected from the group consisting of Andrographis paniculata extract, Nigella sativa extract, Polygonum cuspidatum extract, Piper nigrum extract, 95% tetrahydrocurcuminoids, vitamins and minerals.

### BACKGROUND OF INVENTION

Viruses are the most common agent of respiratory diseases in mammals. Respiratory viruses that most commonly cause infections belong to the class of influenza virus, respiratory syncytial virus, parainfluenza viruses, metapneumovirus, rhinovirus, coronaviruses, adenoviruses, and bocaviruses (Boncristiani et al., Respiratory Viruses. Encyclopedia of Microbiology. 2009: 500-518). The world has seen many endemics and pandemics caused by these respiratory virus, notable ones include Spanish flu, H1N1 endemic, Swine flu, MERS-CoV, Sars-CoV-1 and Sars-CoV-2. The coronavirus disease COVID-19 caused by Sars-CoV-2 caused the worst pandemic the world has ever seen in more than a century. Reports indicate the potential of further pandemics arising due to zoonotic transmission from animals (Lee et al., Emerging threats from zoonotic coronaviruses—from SARS and MERS to 2019-CoV, Journal of Microbiology, Immunology and Infection, Volume 53, Issue 3, June 2020, Pages 365-367). The threats of using these viruses as a tool for biowarfare also looms around.

The mechanism of infection of SARS-CoV-2 consists of the below mentioned steps: (Andrew G. Harrison et al., Mechanisms of SARS-CoV-2 Transmission and Pathogenesis, Trends in Immunology, 2020).
1. Binding of Spike protein to ACE2 on human cells
2. Entry of viral particles into the cells
3. Fusion of the Endosome-Lysosome and release of viral RNA
4. Translation of viral RNA using the host RNA polymerase
5. Viral replication
6. Transcription and translation of structural proteins
7. Assembly and release of viral particles

The binding of spike protein of the virus to the ACE 2 receptor on human cells is the first step in the viral pathogenesis which facilitates the entry of the virus into the cells. The surface spike is composed of S1 subunit, which harbors the receptor binding domain (RBD), and the S2 subunit, which anchors the spike to the viral envelope, following protease activation. The Spike glycoprotein of CoV-2 harbors a Furin cleavage site, at the boundary between its two subunits, S1/S2 (Canrong et al., Furin: A Potential Therapeutic Target for COVID-19, iScience. 2020 Oct. 23; 23(10):101642). Inhibiting Furin can prevent the proteolytic activation of the spike protein thereby preventing the entry virus into the cells via the ACE2 receptors. The importance of Furin and other proteolytic enzymes is provided in the below prior art documents:
1. Wise R J et al., “Expression of a human proprotein processing enzyme: correct cleavage of the von Willebrand factor precursor at a paired basic amino acid site”. Proceedings of the National Academy of Sciences of the United States of America, 1990.
2. Kiefer M C et al., “Identification of a second human subtilisin-like protease gene in the fes/fps region of chromosome 15”. DNA and Cell Biology, 1991.
3. Elisabeth Braun & Daniel Sauter, Furin-mediated protein processing in infectious diseases and cancer Clinical & Translational Immunology 2019; e1073. doi: 10.1002/cti2.1073.
4. Lin et al., Soluble hemojuvelin is released by proprotein convertase-mediated cleavage at a conserved polybasic RNRR site, Blood Cells, Molecules & Diseases, Volume 40, Issue 1, 2008, Pages 122-131
5. Kuninger et al., Pro-protein convertases control the maturation and processing of the iron-regulatory protein, RGMc/hemojuvelin, BMC Biochem 9, 9 (2008). https://doi.org/10.1186/1471-2091-9-9

Currently, scientists all around the world are tirelessly working on finding a suitable drug moiety to manage the infections caused by these viruses and to enhance the immunity. Natural molecules, with anti-viral potential are also being tested to inhibit the growth of virus and to stop the spread. Complementary and alternative medicine (CAM) is generally used in conjunction with conventional medicine to decrease symptoms' severity and promote general well-being (P. M. Barnes, B. Bloom, and R. L. Nahin, “Complementary and alternative medicine use among adults and children: United States, 2007,” Natural Health State Report, vol. 12, pp. 1-23, 2008). More often alternative medicine can be integrated with mainstream healthcare, especially for managing chronic diseases. Since there is an absence of a specific cure for COVID-19, alternative medicine is generally explored to reduce morbidity and complications during the infection. The immune status of patients plays an essential role in the progression of COVID-19 infection. Herbal formulation with an immunomodulatory effect could potentially have a prophylactic effect and may even help as a therapeutic agent for COVID-19-infected patients (M. A. Chowdhury, N. Hossain, M. A. Kashem, M. A. Shahid, and A. Alam, “Immune response in COVID-19: a review,” Journal of Infection and Public Health, vol. 13, no. 11, pp. 1619-1629, 2020). The emergence of variant strains like Mu, lamda, delta, omicron etc stress the need for a balanced immunity. Evidence indicate that supportive or adjunctive therapies can help hasten recovery and reduce severe morbidity and mortality in COVID-19 patients.

Chinese herbal medicines have played an essential role in tackling COVID-19 in China (D. Hong-Zhi, H. Xiao-Ying, M. Yu-Huan, B.-S. Huang, and L. Da-Hui, “Traditional Chinese Medicine: an effective treatment for 2019 novel coronavirus pneumonia (NCP),” Chinese Journal of Natural Medicines, vol. 18, no. 3, pp. 206-210, 2020). Several herbal extracts and formulations are reported to be effective against SARS-CoV-2. The following documents disclose the potential of different plant extracts in the management of viral infections which are herein incorporated by reference:
1. B. Benarba and A. Pandiella, “Medicinal plants as sources of active molecules against COVID-19,” Frontiers in Pharmacology, vol. 11, p. 1189, 2020).
2. J. T. Coon and E. Ernst, “Andrographis paniculata in the treatment of upper respiratory tract infections: a systematic review of safety and efficacy,” Planta Medica, vol. 70, no. 4, pp. 293-298, 2005.
3. A. Basak, S. Cooper, A. G. Roberge, U. K. Banik, M. Chrétien, and N. G. Seidah, “Inhibition of proprotein convertases-1, -7 and furin by diterpines of Andrographis paniculata and their succinoyl esters,” Biochemical Journal, vol. 338, no. Pt 1, pp. 107-113, 1999.
4. T.-H. Shi, Y.-L. Huang, C.-C. Chen et al., “Andrographolide and its fluorescent derivative inhibit the main proteases of 2019-nCoV and SARS-CoV through covalent linkage,” Biochemical and Biophysical Research Communications, vol. 533, no. 3, pp. 467-473, 2020
5. M. Yang, J. Wei, T. Huang et al., “Resveratrol inhibits the replication of severe acute respiratory syndrome coronavirus 2 (SARS-CoV-2) in cultured Vero cells,” Phytotherapy Research, vol. 35, no. 3, pp. 1127-1129, 2021.
6. J. Singh, D. Malik, and A. Raina, “Computational investigation for identification of potential phytochemicals and antiviral drugs as potential inhibitors for RNA-dependent RNA polymerase of COVID-19,” Journal of Biomolecular Structure and Dynamics, pp. 1-16, 2020, doi: 10.1080/07391102.2020.1847688

However, there exists an unmet industrial need for a combination of herbal actives that can be used as a safe adjunctive therapy for the management of viral infections along with standard treatment of care. The present invention herewith discloses compositions comprising ore or more ingredients selected from the group consisting of Andrographis paniculata extract, Nigella sativa extract, Polygonum cuspidatum extract, Piper nigrum extract, 95% tetrahydrocurcuminoids, vitamins and minerals for the management of viral infections.

It is the principle object of the invention to disclose methods and compositions comprising Andrographis paniculata extract standardized to contain not less than 10% andrographolides, Polygonum cuspidatum extract standardized to contain not less than 80% w/w resveratrol, Piper nigrum extract standardized to contain 95% w/w piperine, 95% tetrahydrocurcuminoids, vitamins and minerals for the management of viral infections. The composition further comprises Nigella sativa extract standardized to contain not less than 5% w/w thymoquinone.

It is another object of the invention to disclose methods and compositions comprising Andrographis paniculata extract standardized to contain not less than 10% w/w andrographolides, Polygonum cuspidatum extract standardized to contain not less than 80% w/w resveratrol, Piper nigrum extract standardized to contain 95% w/w piperine, 95% tetrahydrocurcuminoids, vitamins and minerals for preventing the activation of viral spike proteins by inhibiting the activity of proteolytic enzymes. The composition further comprises Nigella sativa extract standardized to contain not less than 5% w/w thymoquinone.

The present invention solves the above-mentioned objects and provides further related advantages.

### SUMMARY OF THE INVENTION

In a most preferred embodiment, the invention discloses a method of preventing the activation of spike proteins in pathogenic mammalian viruses by inhibiting the activity of proteolytic enzymes involved in spike protein activation, said method comprising step of bringing into contact said proteolytic enzymes with a composition comprising Andrographis paniculata extract standardized to contain not less than 10% w/w andrographolides, Polygonum cuspidatum extract standardized to contain not less than 80% w/w resveratrol, Piper nigrum extract standardized to contain 95% w/w piperine, 95% tetrahydrocurcuminoids, vitamins and minerals, to bring about an effect of inhibiting the activity of the enzyme. In a related aspect, the composition further comprises of Nigella sativa extract standardized to contain 2% w/w thymoquinone, about 0.01%-10% w/w thymohydroquinone, about 15%-95% w/w fatty acids, about 0.001%-3% w/w hederagenin or hederin.

In another preferred embodiment, the invention discloses a composition comprising Andrographis paniculata extract standardized to contain not less than 10% w/w andrographolides, Polygonum cuspidatum extract standardized to contain not less than 80% w/w resveratrol, Piper nigrum extract standardized to contain 95% w/w piperine, 95% tetrahydrocurcuminoids, vitamins and minerals for use in preventing the activation of spike proteins in pathogenic mammalian viruses by inhibiting the activity of proteolytic enzymes involved in spike protein activation.

In another most preferred embodiment, the invention discloses a method of therapeutic management of viral infections in mammals, said method comprising step of a) identifying a mammal symptoms of viral infection and b) administering a composition comprising Andrographis paniculata extract standardized to contain not less than 10% w/w andrographolides, Polygonum cuspidatum extract standardized to contain not less than 80% w/w resveratrol, Piper nigrum extract standardized to contain 95% w/w piperine, 95% tetrahydrocurcuminoids, vitamins and minerals, along with standard treatment of care, once a day to bring about a reduction in the severity of viral infection. In a related aspect, the composition further comprises of Nigella sativa extract standardized to contain 2% w/w thymoquinone, about 0.01%-10% w/w thymohydroquinone, about 15%-95% w/w fatty acids, about 0.001%-3% w/w hederagenin or hederin

In another preferred embodiment, the invention discloses a composition comprising Andrographis paniculata extract standardized to contain not less than 10% w/w andrographolides, Polygonum cuspidatum extract standardized to contain not less than 80% w/w resveratrol, Piper nigrum extract standardized to contain 95% w/w piperine, 95% tetrahydrocurcuminoids, vitamins and minerals for use in the therapeutic management of viral infections in mammals. In a related aspect, the composition further comprises of Nigella sativa extract standardized to contain 2% w/w thymoquinone, about 0.01%-10% w/w thymohydroquinone, about 15%-95% w/w fatty acids, about 0.001%-3% w/w hederagenin or hederin.

Other features and advantages of the present invention will become apparent from the following more detailed description, taken in conjunction with the accompanying images, which illustrate, by way of example, the principle of the invention.

### BRIEF DESCRIPTION OF DRAWINGS

FIG. 1 is a flow chart describing the clinical study protocol

FIG. 2A is a graphical representation showing the ordinal scale of disease severity between placebo and ImmuActive groups from screening to the end of study.

FIG. 2B is graphical representation showing the days required to reduce the disease severity by one unit.

### DESCRIPTION OF PREFERRED EMBODIMENTS

In a most preferred embodiment, the invention discloses a method for preventing the activation of spike proteins in pathogenic mammalian viruses by inhibiting the activity of proteolytic enzymes involved in spike protein activation, said method comprising step of bringing into contact said proteolytic enzymes with a composition comprising Andrographis paniculata extract standardized to contain not less than 10% w/w andrographolides, Polygonum cuspidatum extract standardized to contain not less than 80% w/w resveratrol, Piper nigrum extract standardized to contain 95% w/w piperine, 95% tetrahydrocurcuminoids, vitamins and minerals, to bring about an effect of inhibiting the activity of the enzyme. In a related aspect, the composition further comprises of Nigella sativa extract standardized to contain 2% w/w thymoquinone, about 0.01%-10% w/w thymohydroquinone, about 15%-95% w/w fatty acids, about 0.001%-3% w/w hederagenin or hederin. In another related embodiment, the vitamins are selected from the group consisting of Vitamin A, Vitamin C, Vitamin D, Vitamin E, Vitamin K, Vitamin B1 (thiamine), Vitamin B2 (riboflavin), Vitamin B3 (niacin), Pantothenic acid (B5), Biotin (B7), Vitamin B6, Vitamin B12 (cyanocobalamin), Folate (folic acid and B9). In a preferred embodiment, the vitamins are selected from the group consisting of Vitamin C, Vitamin B2 (riboflavin), Vitamin D and Vitamin B6. In another related aspect, the minerals are selected from the group consisting of calcium, phosphorus, magnesium, sodium, potassium, chloride, sulfur, iron, manganese, copper, iodine, zinc, cobalt, fluoride and selenium. In a preferred aspect, the minerals are selected from the group consisting of zinc, copper and selenium. In a related aspect, zinc is present in the form of zinc monomethionine. In another related aspect, copper is present in the form of copper lysinate. In another related aspect, selenium is present in the form of seleno amino acids methylselenocysteine and selenomethionine. In another related aspect, the viruses are selected from the group consisting of Corona Virus, Human immunodeficiency viruses (HIV), Highly pathogenic avian influenza A viruses, Ebola and Marburg viruses, Flaviviruses, Papillomaviruses and Hepatitis B virus. In a preferred aspect, the viruses belong to a class of coronavirus. In a further preferred aspect, the virus is SARS-Cov-2. In another related aspect, the proteolytic enzymes are selected from the group consisting of Furin, cathepsin L, and trypsin-like serine proteases. In related aspect, inhibiting the activation of viral spike protein prevents the entry of the virus into the cell. In another related aspect, the mammalian viruses are human viruses.

In another preferred embodiment, the invention discloses a composition comprising Andrographis paniculata extract standardized to contain not less than 10% w/w andrographolides, Polygonum cuspidatum extract standardized to contain not less than 80% w/w resveratrol, Piper nigrum extract standardized to contain 95% w/w piperine, 95% tetrahydrocurcuminoids, vitamins and minerals for use in preventing the activation of spike proteins in pathogenic mammalian viruses by inhibiting the activity of proteolytic enzymes involved in spike protein activation. In a related aspect, the composition further comprises of Nigella sativa extract standardized to contain 2% w/w thymoquinone, about 0.01%-10% w/w thymohydroquinone, about 15%-95% w/w fatty acids, about 0.001%-3% w/w hederagenin or hederin. In another related embodiment, the vitamins are selected from the group consisting of Vitamin A, Vitamin C, Vitamin D, Vitamin E, Vitamin K, Vitamin B1 (thiamine), Vitamin B2 (riboflavin), Vitamin B3 (niacin), Pantothenic acid (B5), Biotin (B7), Vitamin B6, Vitamin B12 (cyanocobalamin), Folate (folic acid and B9). In a preferred embodiment, the vitamins are selected from the group consisting of Vitamin C, Vitamin B2 (riboflavin), Vitamin D and Vitamin B6. In another related aspect, the minerals are selected from the group consisting of calcium, phosphorus, magnesium, sodium, potassium, chloride, sulfur, iron, manganese, copper, iodine, zinc, cobalt, fluoride and selenium. In a preferred aspect, the minerals are selected from the group consisting of zinc, copper and selenium. In a related aspect, zinc is present in the form of zinc monomethionine. In another related aspect, copper is present in the form of copper lysinate. In another related aspect, selenium is present in the form of seleno amino acids methylselenocysteine and selenomethionine. In another related aspect, the viruses are selected from the group consisting of Corona Virus, Human immunodeficiency viruses (HIV), Highly pathogenic avian influenza A viruses, Ebola and Marburg viruses, Flaviviruses, Papillomaviruses and Hepatitis B virus. In a preferred aspect, the viruses belong to a class of coronavirus. In a further preferred aspect, the virus is SARS-Cov-2. In another related aspect, the proteolytic enzymes are selected from the group consisting of Furin, cathepsin L, and trypsin-like serine proteases. In a related aspect, the activation of viral spike protein prevents the entry of the virus into the cell. In another related aspect, the mammalian viruses are human viruses.

In another most preferred embodiment, the invention discloses a method of therapeutic management of viral infections in mammals, said method comprising step of a) identifying a mammal symptoms of viral infection and b) administering a composition comprising Andrographis paniculata extract standardized to contain not less than 10% w/w andrographolides, Polygonum cuspidatum extract standardized to contain not less than 80% w/w resveratrol, Piper nigrum extract standardized to contain 95% w/w piperine, 95% tetrahydrocurcuminoids, vitamins and minerals, along with standard treatment of care, once a day to bring about a reduction in the severity of viral infection. In a related aspect, the composition further comprises of Nigella sativa extract standardized to contain 2% w/w thymoquinone, about 0.01%-10% w/w thymohydroquinone, about 15%-95% w/w fatty acids, about 0.001%-3% w/w hederagenin or hederin. In another related embodiment, the vitamins are selected from the group consisting of Vitamin A, Vitamin C, Vitamin D, Vitamin E, Vitamin K, Vitamin B1 (thiamine), Vitamin B2 (riboflavin), Vitamin B3 (niacin), Pantothenic acid (B5), Biotin (B7), Vitamin B6, Vitamin B12 (cyanocobalamin), Folate (folic acid and B9). In a preferred embodiment, the vitamins are selected from the group consisting of Vitamin C, Vitamin B2 (riboflavin), Vitamin D and Vitamin B6. In another related aspect, the minerals are selected from the group consisting of calcium, phosphorus, magnesium, sodium, potassium, chloride, sulfur, iron, manganese, copper, iodine, zinc, cobalt, fluoride and selenium. In a preferred aspect, the minerals are selected from the group consisting of zinc, copper and selenium. In a related aspect, zinc is present in the form of zinc monomethionine. In another related aspect, copper is present in the form of copper lysinate. In another related aspect, selenium is present in the form of seleno amino acids methylselenocysteine and selenomethionine. In another related aspect, the viral infection is caused by viruses are selected from the group consisting of Corona Virus, Human immunodeficiency viruses (HIV), Highly pathogenic avian influenza A viruses, Ebola and Marburg viruses, Flaviviruses, Papillomaviruses and Hepatitis B virus. In a preferred aspect, the viruses belong to a class of coronavirus. In a further preferred aspect, the virus is SARS-Cov-2. In another related aspect, the symptoms of viral infection are selected from the group consisting of sneezing, running nose, nasal congestion, cough, fever, loss of smell and taste, malaise, chilliness, headache, myalgias, sore throat, acute respiratory distress, decrease in SpO₂ levels. In another aspect, administration of the composition results in decrease in viral load, decrease in the number of days in the intensive care, decrease in dependence on inhalers or mucolytic agents, enhanced immunity and improves general health. In another related aspect, the standard treatment of care includes administration of antivirals (remdesivir or favipiravir), paracetamol, antibiotics (azithromycin or doxycycline). In another related aspect, the mammal is human. In another related aspect, the composition is formulated along with pharmaceutically/nutraceutically acceptable excipients, adjuvants, diluents or carriers and administered orally in the form of tablets, capsules, syrups, gummies, powders, suspensions, emulsions, chewables, candies or eatables.

In another preferred embodiment, the invention discloses a composition comprising Andrographis paniculata extract standardized to contain not less than 10% w/w andrographolides, Polygonum cuspidatum extract standardized to contain not less than 80% w/w resveratrol, Piper nigrum extract standardized to contain 95% w/w piperine, 95% tetrahydrocurcuminoids, vitamins and minerals for use in the therapeutic management of viral infections in mammals. In a related aspect, the composition further comprises of Nigella sativa extract standardized to contain 2% w/w thymoquinone, about 0.01%-10% w/w thymohydroquinone, about 15%-95% w/w fatty acids, about 0.001%-3% w/w hederagenin or hederin. In a related aspect the composition is administered along with a standard treatment of care, once a day to bring about a reduction in the severity of viral infection. In another related embodiment, the vitamins are selected from the group consisting of Vitamin A, Vitamin C, Vitamin D, Vitamin E, Vitamin K, Vitamin B1 (thiamine), Vitamin B2 (riboflavin), Vitamin B3 (niacin), Pantothenic acid (B5), Biotin (B7), Vitamin B6, Vitamin B12 (cyanocobalamin), Folate (folic acid and B9). In a preferred embodiment, the vitamins are selected from the group consisting of Vitamin C, Vitamin B2 (riboflavin), Vitamin D and Vitamin B6. In another related aspect, the minerals are selected from the group consisting of calcium, phosphorus, magnesium, sodium, potassium, chloride, sulfur, iron, manganese, copper, iodine, zinc, cobalt, fluoride and selenium. In a preferred aspect, the minerals are selected from the group consisting of zinc, copper and selenium. In a related aspect, zinc is present in the form of zinc monomethionine. In another related aspect, copper is present in the form of copper lysinate. In another related aspect, selenium is present in the form of seleno amino acids methylselenocysteine and selenomethionine. In another related aspect, the viruses are selected from the group consisting of Corona Virus, Human immunodeficiency viruses (HIV), Highly pathogenic avian influenza A viruses, Ebola and Marburg viruses, Flaviviruses, Papillomaviruses and Hepatitis B virus. In a preferred aspect, the viruses belong to a class of coronavirus. In a further preferred aspect, the virus is SARS-Cov-2. In another related aspect, the symptoms of viral infection are selected from the group consisting of sneezing, running nose, nasal congestion, cough, fever, loss of smell and taste, malaise, chilliness, headache, myalgias, sore throat, acute respiratory distress, decrease in SpO₂ levels. In another aspect, administration of the composition results in decrease in viral load, decrease in the number of days in the intensive care, decrease in dependence on inhalers or mucolytic agents, enhanced immunity and improves general health. In another related aspect, the standard treatment of care includes administration of antivirals (remdesivir or favipiravir), paracetamol, antibiotics (azithromycin or doxycycline). In another related aspect, the mammal is human. In another related aspect, the composition is formulated along with pharmaceutically/nutraceutically acceptable excipients, adjuvants, diluents or carriers and administered orally in the form of tablets, capsules, syrups, gummies, powders, suspensions, emulsions, chewables, candies or eatables.

The preferred embodiments of the invention are further described in the following illustrative examples

### Example 1: Anti-Viral Formulations

Compositions comprising Andrographis paniculata extract standardized to contain not less than 10% w/w andrographolides, Polygonum cuspidatum extract standardized to contain not less than 80% w/w resveratrol, Piper nigrum extract standardized to contain 95% w/w piperine, 95% tetrahydrocurcuminoids, vitamins and minerals are formulation into two different formulations as Selendrovir™ and Immuactive® disclosed in table 1 and table 2 respectively. The different plant actives are isolated using methods known in the art and standardized to specific ingredients. Alternatively, the different plant extracts are commercially available from Sami-Sabinsa group. The Andrographis paniculata extract standardized to contain not less than 10% w/w andrographolides is available commercially as Panicin™, Polygonum cuspidatum extract standardized to contain not less than 80% w/w resveratrol is available as Resvenox®, Piper nigrum extract standardized to contain 95% w/w piperine is available as BioPerine®, 95% tetrahydrocurcuminoids is available as C3 Reduct®, the Nigella sativa extract is available as Nigellin®.

TABLE 1
Details of Selendrovir ™ Capsule (600 mg)

| Ingredient | Standardised to contain | Weight | |
|---|---|---|---|
| Vitamin C | as ascorbic acid | 250 | mg |
| Vitamin B2 | as riboflavin-5-phosphate | 5 | mg |
| Vitamin B6 | as pyridoxal-5-phosphate | 5 | mg |
| Zinc (Zinbolic ™) | as Zinc monomethionine | 15 | mg |
| Copper | as Copper lysinate | 1 | mg |
| Selenium | as 50-50% L- | 100 | μg |
| | selenocysteine and L- | | |
| | selenomethionine | | |
| Andrographis paniculata | 60% andrographolides | 90 | mg |
| extract (Panicin ™) | | | |
| C3 Reduct ® ODN | 95% | 50 | mg |
| | tetrahydocurcumioids | | |
| Nigella sativa extract | Not less than 5% | 125 | mg |
| (Nigellin ®) | thymoquinone | | |
| Polygonum cuspidatum | Resveratrol | 50 | mg |
| extract (Resvenox ®) | | | |
| Piper nigrum extract | 95% piperine | 5 | mg |
| (BioPerine ®) | | | |

TABLE 2
Immuactive ™ Capsule (500 mg)

| Ingredient | Standardised to contain | Weight | |
|---|---|---|---|
| Zinc (Zinbolic ™) | as Zinc monomethionine | 10 | mg |
| Selenium | as L-selenomethionine | 40 | μg |
| Andrographis paniculata | >90% andrographolides | 50 | mg |
| extract (Panicin ™) | | | |
| C3 Reduct ® ODN | 95% | 100 | mg |
| | tetrahydocurcumioids | | |
| Polygonum cuspidatum | Resveratrol | 50 | mg |
| extract (Resvenox ®) | | | |
| Piper nigrum extract | 95% piperine | 5 | mg |
| (BioPerine ®) | | | |

### Example 2: Furin Inhibition Potential of Anti-Viral Formulations

The anti-viral formulations—Selendrovir™ and Immuactive™ were tested for inhibiting the Furin enzyme to prevent the activation of the spike protein of SARS-CoV-2.

Method

Materials: Recombinant human Furin protein, Substrate—p-Glu-Arg-Thr-Lys-Arg-AMC—[ERTKR (SEQ ID 1) linked to amido methylcoumarin]

Principle

pGlu-Arg-Thr-Lys-Arg-7-amido-4-methylcoumarin was used as a substrate and activity was measured by the fluorescence generated by the release of amino-4-methylcoumarin.

Protocol

Enzyme (1 μg/ml) was mixed with 25 μM substrate in the presence of different concentrations of anti-viral formulation. Fluorescence of AMC was recorded at excitation and emission wavelengths of 380 nm and 460 nm respectively in kinetic mode for 20 minutes. Percentage inhibition was calculated using Enzyme and substrate activity as reference.

Results

The furin inbition of Selendrovir™ is presented in table 3. The composition inhibited Furin with an IC₅₀ of 41.02 μg/ml.

TABLE 3
Furin inhibition potential of Selendrovir ™

| | Concentration μg/ml | % Inhibition |
|---|---|---|
| Selendrovir ™ | 200 | 71.980 |
| | 50 | 54.712 |
| | 12.5 | 31.253 |

| | |
|---|---|
| IC50 | 41.02 μg/ml. |

The furin inhibition of Immuactive™ is presented in table 4. The composition inhibited Furin with an IC₅₀ of 41.02 μg/ml.

| | Concentration μg/ml | % Inhibition |
|---|---|---|
| Immuactive ™ | 250 | 84.55 |
| | 125 | 77.00 |
| | 62.5 | 70.24 |
| | 31.25 | 46.56 |

| | |
|---|---|
| IC50 | 33.13 μg/ml. |

Both the compositions inhibited furin and thus will be effective as an anti-viral agent. The Immuactive™ composition was effective in inhibiting the enzyme furin compared to Selendrovir™.

### Example 3: Efficacy and Safety of ImmuActive™ for COVID-19 Patients

The formulation containing curcuminoids, andrographolides, resveratrol, selenium, zinc, and piperine-ImmuActive™ was evaluated along with the standard treatment of care in COVID-19 patients.

Methods

The study was conducted as a randomized, double-blind, placebo-controlled, multicenter, two-arm, prospective design. The efficacy and safety of ImmuActive™ were assessed as an adjunct therapy for COVID-19 patients up to a maximum of 28 days or discharge from hospital/COVID-19 care center or transfer to ICU, whichever was earlier in comparison to placebo.

Adult (18-50 years) male and female COVID-19-positive patients with or without comorbid conditions such as diabetes and hypertension with BMI≤35 kg/m2 were included in the study. The subjects who tested positive for COVID-19 by RT-PCR were enrolled within 48 hours. The enrolled patients had an ordinal scale score, less than or equal to 3, with the requirement of hospitalization or admission to the isolation ward, but stable with peripheral capillary oxygen saturation >94% on room air as described in the Ministry of Ayurveda, Yoga and Naturopathy, Unani, Siddha and Homoeopathy (AYUSH), Govt. of India, guidelines for designing COVID-19 clinical studies.

Asymptomatic COVID-19-positive patients with an ordinal scale of >3, under parenteral nutrition or tube feeding, or admitted to isolation ward or hospitalized for >48 hours of confirmed COVID-19-positive test were excluded from the study. Patients on ventilator support, with uncontrolled and unstable comorbidities, having a history of chronic lung disease, active malignancy, chronic kidney disease, and chronic liver disease, those who are immunocompromised, or those on immunosuppressants, allergic to investigational products, participating in another clinical study including macro/micro/any other forms of dietary supplements/multivitamins or oral nutrition supplements as well as pregnant and lactating females were excluded from the study.

Written informed consent was taken from all the subjects before enrollment in the study. The trial was conducted following the Declaration of Helsinki, the International Conference on Harmonization Guidelines for Good Clinical Practice, and applicable local regulations. The study included three centers, Prakriya Hospital (Bangalore), People Tree Hospital (Bangalore), and Apollo Hospital (Chennai), after approval from their respective Institutional Ethics Committee. The trial was registered prospectively on the Clinical Trial Registry of India (CTRI) with the registration number CTRI/2020/09/027841. The standard care of treatment was administered as per hospital treatment protocol and the respective state government's recommendations for managing COVID-19 patients. The treatment included antiviral (remdesivir and favipiravir), paracetamol, antibiotics (azithromycin/doxycycline), and multivitamins.

ImmuActive™ 500 mg capsule was administered orally to subjects once daily after breakfast in the morning. Efficacy was evaluated for 28 days or discharge from hospital/COVID-19 care center or transfer to ICU, whichever was earlier. The ordinal clinical severity scale was assessed every day until the negative RT-PCR test/discharge from the hospital or admission to ICU or a maximum of 28 days in both the intervention and control arm (WHO, R&D Blueprint Novel Coronavirus COVID-19 Therapeutic Trial Synopsis, WHO, Geneva, Switzerland, 2021). The scale has five different patient statuses: uninfected, ambulatory, hospitalized with mild disease, hospitalized with severe disease, and death (Table 5). The score was recorded as 0 when no clinical or virological evidence of infection was observed. Hospitalized with mild disease was further characterized as no limitation of activities (1), limitation of activities (2), and no oxygen therapy (3). The third scale of severe infection was characterized as oxygen by mask or nasal prongs (4), noninvasive ventilation or high-flow oxygen (5), intubation and mechanical ventilation (6), and ventilation+additional organ support-pressors, RRT, and ECMO (7). Death was given a score of 8. In the present study, subjects with less than or equal to a score of three were included. The mean change in ordinal scale from baseline to final visit was compared between ImmuActive and placebo. Further, the mean time required to reduce the scale by one unit was compared between the two groups.

TABLE 5
Ordinal scale for clinical improvement.

| Sno. | Patient State | Descriptor | Score |
|---|---|---|---|
| 1 | Uninfected | Uninfected; no viral RNA detected | 0 |
| 2 | Ambulatory | No limitation activities | 1 |
| | | Limitation of activities | 2 |
| 3 | Hospitalized | Hospitalized; no oxygen therapy | 3 |
| | mild | Oxygen by mask or nasal prongs | 4 |
| | disease | | |
| 4 | Hospitalized | Noninvasive ventilation or high- | 5 |
| | severe | flow oxygen | |
| | disease | Intubation or mechanical | 6 |
| | | ventilation | |
| | | Ventilation + additional organ | 7 |
| | | support-pressors, RRT, and | |
| | | ECMO | |
| 5 | Dead | Death | 8 |

RNA: ribonucleic acid; RRT: renal replacement therapy; ECMO: extracorporeal membrane oxygenation.

Modified Jackson Symptom Severity Score was evaluated using a subjective self-reporting questionnaire for eight symptoms, which included sneezing, nasal discharge, nasal congestion, sore throat, malaise, fever, cough, and headache. Subjects rated each of their symptoms as follows: absent (0), mild (1), moderate (2), or severe (3). Subjects were assessed on randomization and the RT-PCR testing days until a negative RT-PCR result was established.

COVID-19 QOL questionnaires included six questions regarding the quality of the life of an individual. The QOL was recorded on the day of randomization, on day 6, on days when the RT-PCR test was conducted, and on the day of discharge/admission to ICU, whichever was earlier. Subjects were evaluated for their overall quality of life, mental health, physical health, and personal safety. The mean number of days required to turn RT-PCR negative and the number of days of hospitalization were compared between the intervention and placebo arm. The safety of the subjects through the incidence of adverse events was evaluated throughout the study period.

Statistical Analysis

The sample size was derived using population size, confidence level, and marginal error. The sample size was calculated to be 80 based on a confidence level of 95% and a marginal error of 5%. Allowing for a 25% dropout rate, the required sample size for recruitment was 100 in 1:1 ratio between two study groups (i.e., 50 per treatment group). The continuous variables are presented as descriptive statistics of n, mean, standard deviation, and minimum and maximum values, whereas the categorical variables are presented as frequencies and percentages. A two-tailed independent samples t-test was performed to compare the treatment groups (ImmuActive™ and placebo) to present the efficacy endpoint data. A two-tailed paired t-test was performed to compare the baseline data with the end of the study data for individual treatment groups. The efficacy parameters, namely, ordinal scale, modified Jackson's Symptom Score, and COVID-19 quality of life questionnaire, were compared between active and placebo arms. A chi-square test was conducted for categorical variables. The differences in results obtained for the individual treatment groups between baseline and end of the study are presented as mean change from baseline and the p value derived through a two-tailed paired t-test. The two treatment groups were also compared in terms of the number of days taken for a change in disease severity on an ordinal scale by one, and the p value was derived through a two-tailed independent samples t-test.

A descriptive comparison of efficacy endpoints like the number of days of hospitalization and the number of days to negative RT-PCR reports is presented. A value of <0.05 was considered statistically significant. Two decimal places are retained for all values. All the statistical analyses in the study were conducted using the STATA software version 16.0.

Results

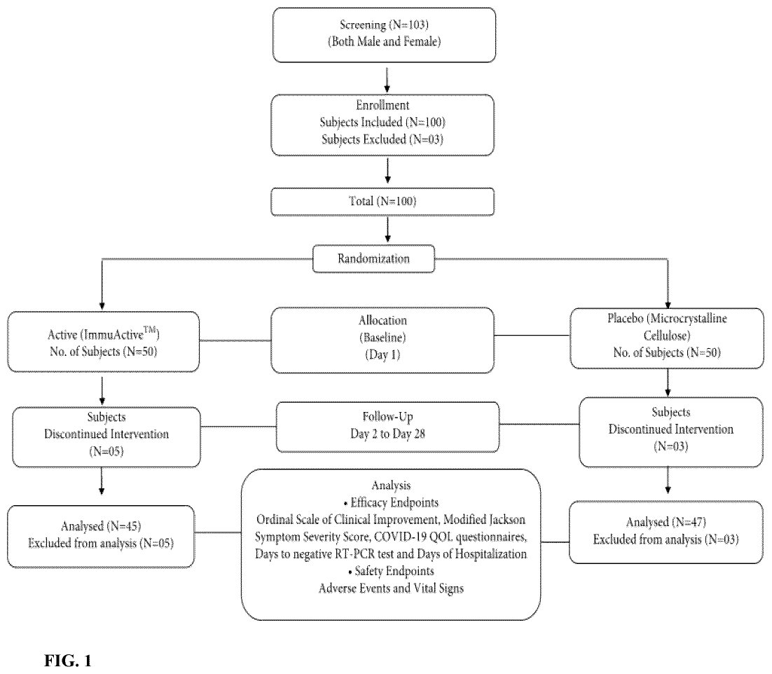

A total of 103 subjects were screened, and 100 (71 males and 29 females) were enrolled and randomized to N=50 in the placebo and active arms. Eight subjects withdrew from the study, and 92 subjects (47 in placebo and 45 in ImmuActive arm) completed the study (FIG. 1).

The mean age was 38.16 years among all subjects, 39.04 years in the active group, and 37.28 years in the placebo group at baseline. Patients' demographics were comparable between the groups. Vital signs were measured as a part of safety analysis, and no abnormal or out-of-range values were observed. The temperature ranged from 96.5 to 102.2° F., and SpO2 ranged from 95 to 99%. The detailed demographics and vital signs are presented in Table 6.

TABLE 6
Demographics and vital signs

| | ImmuActive ™ | Placebo | |
|---|---|---|---|
| Parameters | (N = 50) | (N = 50) | P value |
| Age (years) | 39.04 ± 7.70 | 37.28 ± 7.40 | 0.159 (NS) |
| Height (cm) | 167.32 ± 6.40 | 166.18 ± 6.30 | 0.785 (NS) |
| Weight (kg) | 67.96 ± 6.25 | 70.08 ± 7.56 | 0.304 (NS) |
| BMI (kg/m²) | 24.32 ± 1.91 | 25.40 ± 2.14 | 0.064 (NS) |
| Systolic blood | 122.42 ± 7.54 | 123.30 ± 10.28 | 0.396 (NS) |
| pressure | | | |
| (mmHg) | | | |
| Diastolic blood | 79.30 ± 7.72 | 80.82 ± 7.82 | 0.496 (NS) |
| pressure | | | |
| (mmHg) | | | |
| Body | 98.43 ± 0.96 | 98.35 ± 0.72 | 0.678 (NS) |
| temperature | | | |
| (° F.) | | | |
| Pulse rate | 90.56 ± 7.49 | 92.02 ± 7.32 | 0.474 (NS) |
| (beats/min) | | | |
| Oxygen | 96.90 ± 0.99 | 97.06 ± 1.15 | 0.293 (NS) |
| saturation | | | |
| (SpO₂) % | | | |

NS—not significant

Primary Clinical Outcomes

Change in Ordinal Scale

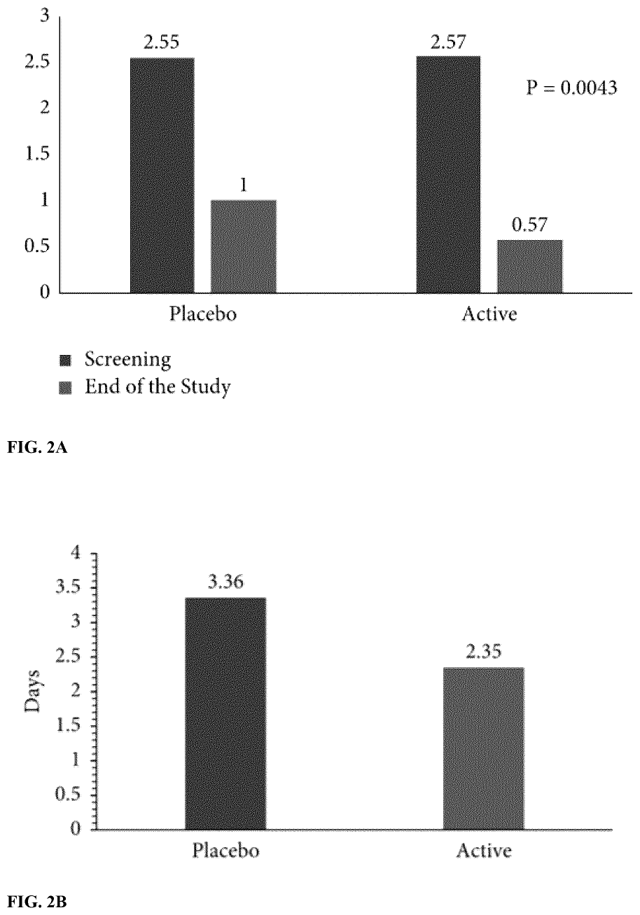

The ordinal scale of disease severity significantly decreased from screening to the end of the study
in the ImmuActive (2.57 to 0.57, N=45) group compared to the placebo group (2.55 to 1.0, N=47) with a p value of 0.0043 (FIG. 2A). ImmuActive was found to have a significantly better (p=0.033) therapeutic response compared to placebo for the mean duration (days) required to reduce disease severity on the ordinal scale by 1 unit. Among the subjects receiving ImmnuActive treatment, the mean duration was 2.35 days, whereas, in subjects receiving placebo, the mean duration was 3.36 days (FIG. 2).

Secondary Clinical Outcomes

Clinical Evidence of Infection

At the end of the study, 19 patients (42.2%) in the ImmuActive group had no clinical or virological evidence of infection, and the ordinal score was recorded as 0, compared to 11 (23.4%) in the placebo group (=0.03), which was statistically significant (Table 7).

TABLE 7
Secondary Clinical outcomes

| | Active | | Placebo | | |
|---|---|---|---|---|---|
| | Number of | | Number of | | |
| Parameter | patients | Percentage | patients | Percentage | p Value |

Ordinal scale

| | | | | | |
|---|---|---|---|---|---|
| 0 | 19 | 44.22 | 11 | 23.40 | 0.03 |
| 1 | 26 | 57.78 | 18 | 38.30 | |
| 2 | 0 | 0 | 16 | 34.04 | |
| 3 | 0 | 0 | 1 | 2.13 | |
| 4 | 0 | 0 | 1 | 2.13 | |

Number of days in hospital

| | | | | | |
|---|---|---|---|---|---|
| Less | 1 | 2.22 | 0 | | NS |
| than 6 | | | | | |
| 6-10 | 4 | 97.78 | 42 | 89.36 | |
| >10 | 0 | | 5 | 10.64 | |

No. of days of RT-PCR negative

| | | | | | |
|---|---|---|---|---|---|
| 6 days | 29 | 64.44 | 27 | 57.45 | NS |
| 7-10 | 1 | 2.22 | 7 | 14.89 | |
| 10-14 | 15 | 33.33 | 11 | 23.40 | |
| >14 | 0 | 0.00 | 2 | 4.26 | |

Patients with ordinal scale of “0”: no clinical evidence of infection.
NS: not significant.

Days of Hospitalization

Length of hospital stay was marginally lower in the ImmuActive group (7.41 f 1.79, median 6.5) than the placebo group (7.74±2.35, median 7), and the difference was not statistically significant. The maximum number of days of hospitalization was ten days (median 6.5 (5-10)) in ImmuActive group and 15 days (median 7 (6-15)) in the placebo group. ImmuActive as an adjunct therapy reduced the maximum number of days in hospital by five days. Similarly, the number of days in hospital reduced in 10% population compared to the placebo group (Table 7).

The Number of Days to Negative RT-PCR Report

The mean number of days to negative RT-PCR in the ImmuActive arm was 7.43±2.11 and 7.89±3.68 in the placebo arm. In the ImmuActive arm, 29 patients turned RT-PCR negative within six days, and the rest 16 turned negative by 14 days. In contrast, 27 patients in placebo turned negative in 6 days and 18 patients within 14 days, and two of them took more than 14 days to turn negative. The maximum number of days required to turn RT-PCR negative was 24 days in the placebo arm compared to 14 days in the active arm. Two patients from the placebo arm took more than 14 days to turn negative in the RT-PCR test (Table 7).

Modified Jackson's Symptom Severity Score

Modified Jackson's Symptom Severity Score significantly decreased at the end of the study in both ImmuActive (4.98±2.18 to 1.36±1.71; p<0.0001) and placebo groups (5.11±2.29 to 1.70±1.68; p<0.0001) in comparison with the screening visit. However, the mean change in score was not statistically significant when compared between ImmuActive and placebo (Table 8).

TABLE 8
Modified Jackson's Symptom Severity Score.

| | | | |
|---|---|---|---|
| | Active | Placebo | |
| | (ImmuActive ™) | (microcrystalline | |
| | group | cellulose) group | |
| Parameter | (N = 45) | (N = 47) | p value |

Screening visit (visit 1)

| | | | |
|---|---|---|---|
| Mean ± SD | 4.98 ± 2.18 | 5.11 ± 2.29 | 0.7812(NS) |
| Minimum | 2.00 | 1.00 | |
| Maximum | 10.00 | 11.00 | |

End visit

| | | | |
|---|---|---|---|
| Mean ± SD | 1.36 ± 1.71 | 1.70 ± 1.68 | 0.3413(NS) |
| Minimum | 0.00 | 0.00 | |
| Maximum | 8.00 | 5.00 | |
| Mean change | −3.62 | −3.41 | |
| from | | | |
| baseline | | | |
| p value | <0.0001 | <0.0001 | |

NS, not significant;.*P < 0.05

COVID-19 Quality of Life Questionnaire

A significant change in the mean of COVID-19 quality of life questionnaire was observed at the end in both ImmuActive (9.51±3.25 to 6.82±1.79; p<0.0001) and placebo (9.06±2.98 to 6.83±1.30; p<0.0001) groups in comparison with their screening visit (Table 4). Mean change in the COVID-19 quality of life questionnaire was similar in both the ImmuActive (−2.69) and placebo arms (−2.24) at the end visit (Table 9). The number of patients requiring antiviral therapy was comparatively higher in the placebo group.

TABLE 9
COVID-19 quality of life questionnaire.

| | | | |
|---|---|---|---|
| | Active | Placebo | |
| | (ImmuActive ™) | (microcrystalline | |
| | group | cellulose) group | |
| Parameter | (N = 45) | (N = 47) | p value |

Screening visit (visit 1)

| | | | |
|---|---|---|---|
| Mean ± SD | 9.51 ± 3.25 | 9.06 ± 2.98 | 0.4903(NS) |
| Minimum | 6.00 | 6.00 | |
| Maximum | 23.00 | 21.00 | |

End visit

| | | | |
|---|---|---|---|
| Mean ± SD | 6.82 ± 1.79 | 6.83 ± 1.30 | 0.9757 (NS) |
| Minimum | 6.00 | 6.00 | |
| Maximum | 17.00 | 11.00 | |
| Mean change | −2.69 | −2.24 | |
| from | | | |
| baseline | | | |
| p value | <0.0001 | <0.0001 | |

NS, not significant; *statistically significant

Safety Analysis

A total of 100 subjects were enrolled, and 92 subjects completed the study; out of the 100 subjects, none reported adverse events in both ImmuActive and placebo groups. Eight subjects withdrew from the study for personal reasons.

To conclude the clinical study suggests that supplementation with ImmuActive with the standard treatment reduced the COVID-19 infection symptoms. The supplementation also significantly reduced the number of days spent in the hospital and the number of days required to turn viral negative by RT-PCR test. Significant improvement in ordinal scale suggests that the patients supplemented with ImmuActive moved from hospitalized stage to ambulatory, with no limitations on their activity compared to the placebo arm. Further, both Selendrovir™ and Immuactive™ increases immunity and improves general health of the patients. Since there are no side effects, it can be effectively used a supplement to boost immune health and prevent further infections.

Other modifications and variations of the invention will be apparent to those skilled in the art from the foregoing disclosure and teachings. Thus, while only certain embodiments of the invention have been specifically described herein, it will be apparent that numerous modifications may be made thereto without departing from the spirit and scope of the invention and is to be interpreted only in conjunction with the appended claims.

We claim:

1. A composition comprising Andrographis paniculata extract standardized to contain not less than 10% w/w andrographolides, Polygonum cuspidatum extract standardized to contain not less than 80% w/w resveratrol, Piper nigrum extract standardized to contain 95% w/w piperine, 95% tetrahydrocurcuminoids, vitamins and minerals.

2. The composition as in claim 1, wherein the composition further comprises Nigella sativa extract standardized to contain 2% w/w thymoquinone, about 0.01%-10% w/w thymohydroquinone, about 15%-95% w/w fatty acids, about 0.001%-3% w/w hederagenin or hederin.

3. The composition as in claim 1, wherein the vitamins are selected from the group consisting of Vitamin C, Vitamin B2 (riboflavin), Vitamin D and Vitamin B6.

4. The composition as in claim 1, wherein the minerals are selected from the group consisting of zinc, copper and selenium.

5. The composition as in claim 4, wherein zinc is present in the form of zinc monomethionine.

6. The composition as in claim 4, wherein copper is present in the form of copper lysinate.

7. The composition as in claim 4, wherein selenium is present in the form of methylselenocysteine and/or selenomethionine.

8. The composition as in claim 1, wherein the said composition is effective in preventing the activation of spike proteins in pathogenic mammalian viruses by inhibiting the activity of proteolytic enzymes involved in spike protein activation.

9. The composition as in claim 8, wherein the viruses are selected from the group consisting of Corona Virus, Human immunodeficiency viruses (HIV), Highly pathogenic avian influenza A viruses, Ebola and Marburg viruses, Flaviviruses, Papillomaviruses and Hepatitis B virus.

10. The composition as in claim 8, wherein the virus is SARS-Cov-2.

11. The composition as in claim 8, wherein the proteolytic enzymes are selected from the group consisting of Furin, cathepsin L, and trypsin-like serine proteases.

12. The composition as in claim 8, wherein inhibiting the activation of viral spike protein prevents the entry of the virus into the cell.

13. The composition as in claim 8, wherein the mammalian viruses are human viruses.

14. The composition as in claim 1, wherein said composition is effective in the therapeutic management of viral infections in mammals.

15. The composition as in claim 14, wherein the composition is administered along with a standard treatment of care, once a day to bring about a reduction in the severity of viral infection.

16. (canceled)

17. (canceled)

18. (canceled)

19. (canceled)

20. (canceled)

21. The composition as in claim 14, wherein the viruses are selected from the group consisting of Corona Virus, Human immunodeficiency viruses (HIV), Highly pathogenic avian influenza A viruses, Ebola and Marburg viruses, Flaviviruses, Papillomaviruses and Hepatitis B virus.

22. The composition as in claim 14, wherein the virus is SARS-Cov-2.

23. The composition as in claim 14, wherein administration of the composition results in decrease in viral load, decrease in the number of days in the intensive care, decrease in dependence on inhalers or mucolytic agents, enhanced immunity and improves general health.

24. The composition as in claim 15, wherein the standard treatment of care includes administration of antivirals (remdesivir or favipiravir), paracetamol, antibiotics (azithromycin or doxycycline).

25. The composition as in claim 14, wherein the mammal is human.

26. The composition as in claim 2, wherein the composition is formulated with pharmaceutically/nutraceutically acceptable excipients, adjuvants, diluents or carriers and administered orally in the form of tablets, capsules, syrups, gummies, powders, suspensions, emulsions, chewables, candies or eatables.
